(19) United States
(12) Patent Application Publication
(10) Pub. No.: US 2022/0241009 A1
(43) Pub. Date: Aug. 4, 2022

(54) IRRIGATION IN ASSOCIATION WITH PULSED ELECTRIC FIELD ABLATION

(71) Applicant: Medtronic, Inc., Minneapolis, MN (US)

(72) Inventors: Brian T. Howard, Minneapolis, MN (US); Timothy G. Laske, Shoreview, MN (US); Gregory Scott Brumfield, Delaware, OH (US)

(21) Appl. No.: 17/584,770

(22) Filed: Jan. 26, 2022

Related U.S. Application Data
(60) Provisional application No. 63/144,005, filed on Feb. 1, 2021.

(51) Int. Cl. A61B 18/14

(52) U.S. Cl. CPC A61B 18/1482; A61B 2018/1465; A61B 2018/00083; A61B 2218/002; A61B 2018/1467

(57) ABSTRACT

A medical device is configured to deliver pulsed electric field (PEF) energy to tissue and includes an elongated shaft having a proximal portion and a distal portion. A balloon is coupled to the distal portion of the elongated shaft. A plurality of electrodes is disposed on an outer surface of the balloon and configured to apply PEF energy to tissue. The balloon defines one or more irrigation channels proximate to or on the plurality of electrodes, the one or more irrigation channels being configured to irrigate at least one of the plurality of electrodes.

### CROSS-REFERENCE TO RELATED APPLICATION

This application claims the benefit of U.S. Application Ser. No. 63/144,005, filed Feb. 1, 2021.

### FIELD

The present technology is generally related to devices, systems, and methods with irrigation for ablation treatments.

### BACKGROUND

Medical procedures such as cardiac ablation using one or more energy modalities are frequently used to treat conditions such as atrial fibrillation and ventricular tachycardia. However, complications may arise during these procedures related to the use of the various energy modalities. These complications are observed currently with thermal energy delivery, such as radiofrequency ablation, which may cause collateral damage to non-targeted tissue including blood, nerve, and organ tissue, for example. Further, thermal energy application by itself may not cause adequate lesion formation in the targeted tissue such as the myocardium and, therefore, the underlying condition can persist. Certain energy modalities, such as pulsed electric field (PEF) ablation, however, use electric fields to disrupt cellular membranes and these electric fields are delivered in short bursts that are less likely to cause thermal damage to non-target tissue. However, it may still be challenging to create adequate lesions, such as fully circumferential, contiguous, and/or transmural lesions. The electric field itself is established between conductive elements such as electrodes and flows current through the target tissue acting as a resistive medium, and this necessarily results in energy dissipation or temperature rise in the tissue.

Ablation may be affected with PEF without imparting sufficient energy to cause thermal damage as this is an identified risk of radiofrequency ablation. In general terms, to affect a larger region of tissues, application of higher energies for PEF may be used to more thoroughly treat a targeted region while the tradeoff is an increase in the dissipated energy or corresponding temperature rise in the tissue. Mitigations to this effect may increase the energy that may be delivered with PEF while reducing the risk of thermal damage. In particular, edge effects, such as increased current being directed toward the edges of the electrodes may cause the edges of the electrodes to exchange heat with the tissue beyond a desired amount making the mitigation of thermal effects more important at these regions.

### SUMMARY

The techniques of this disclosure generally relate to irrigation for pulsed electric field (PEF) ablation treatments to mitigate the effect of increasing the energy that may be delivered with PEF by, for example, reducing the risk of thermal damage to unintended tissue or reducing the risk of the formation of char or coagulum.

In one embodiment, a medical device is configured to deliver pulsed electric field (PEF) energy to tissue and includes an elongated shaft having a proximal portion and a distal portion. An expandable element is coupled to the distal portion of the elongated shaft and the expandable element has an outer surface and an inner surface opposite the outer surface. A plurality of electrodes are disposed on the outer surface of the expandable element and the plurality of electrodes are configured to apply energy to tissue. The expandable element comprising one or more irrigation channels proximate to or on the plurality of electrodes, the one or more irrigation channels being configured to irrigate at least one of the plurality of electrodes.

In another aspect of this embodiment, the one or more channels are disposed around a perimeter of each of the plurality of electrodes.

In another aspect of this embodiment, an irrigant is disposed within the one or more irrigation channels and the irrigant only flows toward respective electrodes which are energized during the delivery of energy to tissue.

In another aspect of this embodiment, the irrigant is cooler than an ambient temperature of blood.

In another aspect of this embodiment, the irrigant has a lower conductivity than blood.

In another aspect of this embodiment, the irrigant has a higher conductivity than blood.

In another aspect of this embodiment, a perimeter of each of the plurality of electrodes has a higher thermal conductivity compared to a remainder of the each one of the plurality of electrodes to reduce edge effects and heating.

In another aspect of this embodiment, a perimeter of each of the plurality of electrodes has a lower electrical conductivity compared to a remainder of the each one of the plurality of electrodes to reduce edge effects and heating.

In another aspect of this embodiment, the irrigant is a contrast media that is visible using medical imaging under ultrasound or fluoroscopy to confirm irrigation.

In another aspect of this embodiment, the one or more irrigation channels are configured to selectively irrigate at least one of the plurality of electrodes based upon a desired flow rate, a particular timing, or the electrode temperature.

In one embodiment, a medical system is configured to deliver pulsed electric field (PEF) energy to tissue and includes a medical device, the medical device includes an elongated shaft having a proximal portion and a distal portion. A balloon having an outer surface and an inner surface opposite the outer surface is coupled to the distal portion of the elongated shaft. A plurality of electrodes is disposed on an outer surface of the balloon and configured to apply PEF energy to the tissue and each electrode has a perimeter. The balloon including one or more irrigation channels around the perimeter of each of the plurality of electrodes, the one or more irrigation channels being configured to selectively irrigate the plurality of electrodes. A fluid source is in communication with the one or more irrigation channels. A controller is in communication with the fluid source and the medical device, the controller is configured to deliver PEF energy to the plurality of electrodes and to modulate a delivery of a fluid from the fluid source to the one or more irrigation channels based upon preset parameters derived from prior deliveries of PEF energy to the tissue.

In another aspect of this embodiment, the preset parameters derived from prior deliveries of PEF energy to the tissue include at least one from the group consisting of: temperature rise, impedance change, quantity of fluid delivered, pressure of the irrigation channel, measured flow rate, change in delivered current over a period of PEF delivery, and total energy expenditure of energy source for PEF energy delivery.

In another aspect of this embodiment, the controller is further configured to modulate an amount of fluid delivered to the one or more irrigation channels based at least in part on preselected PEF ablation parameters.

In another aspect of this embodiment, the preselected PEF parameters include at least one from the group consisting of applied voltage, pulse width, cycle lengths, number of applied pulses per application, number of applications, and a selection of which ones of the plurality of electrodes are engaged in PEF delivery.

In another aspect of this embodiment, the controller is further configured to modify a temperature of the fluid in the fluid source.

In another aspect of this embodiment, the fluid source includes at least two type of fluids.

In another aspect of this embodiment, the fluid in the fluid source has a net negative charge.

In another aspect of this embodiment, the plurality of electrodes includes an anti-thrombogenic coating.

In another aspect of this embodiment, the fluid in the fluid source is configured to increase a vulnerability of the tissue to PEF energy.

In one aspect, a method of delivering pulsed electric field (PEF) energy to tissue includes advancing a distal portion of a medical device proximate the tissue, the medical device includes: a balloon at the distal portion; a plurality of electrodes disposed on an outer surface of the balloon and configured to deliver PEF energy; and a plurality of irrigation channels disposed around a perimeter of each of the plurality of electrodes. The method further includes selectively irrigating at least one of the plurality of electrodes.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a perspective view of an exemplary pulsed electric field delivery system;

FIG. 2 is a side view of the expandable element shown in FIG. 1 with selective irrigation channels;

FIG. 3 is a side view of the expandable element shown in FIG. 2 with select electrodes irrigated;

FIG. 4A is a top view of a select exemplary pair of electrodes showing fluid ports around each electrode;

FIG. 4B is a top view of a select exemplary pair of electrodes showing fluid ports around each electrode;

FIG. 4C is a top view of a select exemplary pair of electrodes showing fluid ports around a portion each electrode;

FIG. 4D is a top view of a select exemplary pairs of electrodes showing fluid ports around a portion each electrode;

FIG. 5 is a top view of an exemplary electrode with fluid ports disposed around an edge of the electrode;

FIG. 6A is a top view of select exemplary electrodes showing fluid ports surrounding the electrode with opposite polarity;

FIG. 6B is a top view of select exemplary electrodes showing a fluid port surrounding the electrode with opposite polarity;

FIG. 7A is a top view of select exemplary electrodes showing a fluid port surrounding adjacent electrodes of opposite polarity;

FIG. 7B is a top view of select exemplary electrodes showing a fluid port surrounding adjacent electrodes of opposite polarity and partially surrounding additional electrodes; and

FIG. 8 is a method of use of the exemplary pulsed electric field delivery system.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

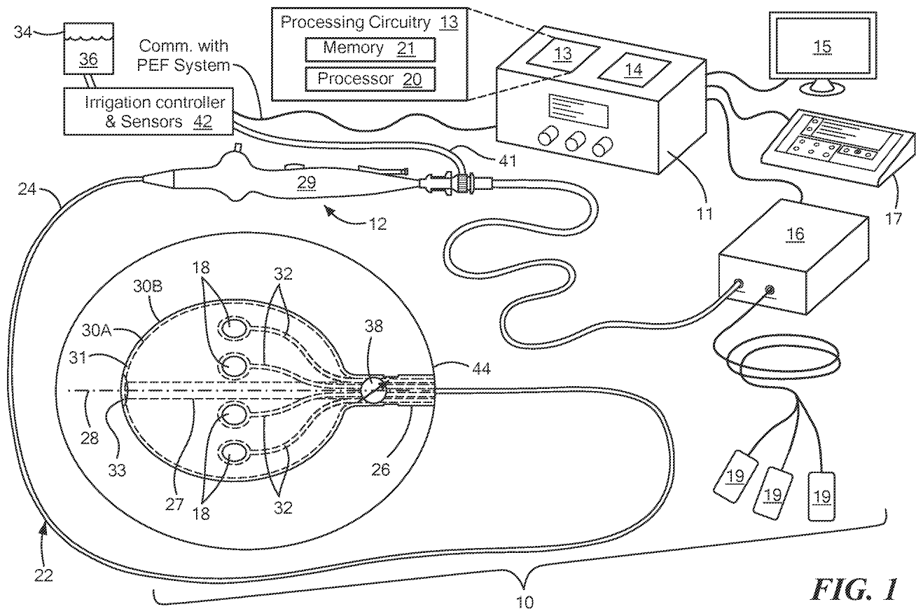

Referring now to the drawing figures in which like reference designations refer to like elements, a first exemplary embodiment of a medical system constructed in accordance with the principles of the present invention is shown in FIG. 1, generally designated as “10.” The system 10 may generally include a medical device 12, such as a catheter, that may be coupled directly to an energy supply 14, such as a pulsed electric field energy generator. The energy supply 14 may include an energy control, delivering, and monitoring system. Alternatively, the system 10 may be coupled to a device electrode distribution system 16 (which may also be referred to herein as a “catheter electrode distribution system” or “CEDS”). The energy supply 14 may be within or in electrical communication with a controller 11 having processing circuitry 13 that may further include or be in electrical communication with one or more other system components, such as one or more displays 15, the CEDS 16, user input devices 17, surface electrodes 19, and the like.

For simplicity, all system components other than the medical device 12 may be collectively referred to as being part of the controller 11. In addition to being configured to deliver ablation energy, such as pulsed electric field energy, a plurality of electrodes 18 may also be configured to perform diagnostic functions, such as to collect intracardiac electrograms (EGM) and/or monophasic action potentials (MAPs) as well as performing selective pacing of intracardiac sites for diagnostic purposes or providing connection paths to other electrophysiology monitoring systems for such tasks.

The controller 11 may be a remote controller that includes the processing circuitry 13 configured to operate and control the various functions of the system 10. Alternatively, in some configurations the user input device 17 may include the processing circuitry 13. In one or more embodiments, the processing circuitry 13 may include a processor 20 and a memory 21. In particular, in addition to or instead of a processor, such as a central processing unit, and memory, the processing circuitry 13 may comprise integrated circuitry for processing and/or control, e.g., one or more processors and/or processor cores and/or FPGAs (Field Programmable Gate Array) and/or ASICs (Application Specific Integrated Circuitry) adapted to execute instructions. The processor 20 may be configured to access (e.g., write to and/or read from) the memory 21, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory).

The processing circuitry 13 may be configured to control any of the methods and/or processes described herein and/or to cause such methods, and/or processes to be performed, e.g., by the controller 11. Processor 20 corresponds to one or more processors 20 for performing functions described herein. The memory 21 is configured to store data, programmatic software code and/or other information described herein. In some embodiments, the software may include instructions that, when executed by the processor 20 and/or processing circuitry 13 causes the processor 20 and/or processing circuitry 13 to perform the processes described herein with respect to controller 11. For example, processing circuitry 13 of the controller 11 may be configured to perform one or more functions described herein such as with respect to methods and systems described in more detail herein.

Further, the medical device 12 may include one or more diagnostic or treatment regions for the energetic, therapeutic, and/or investigatory interaction between the medical device 12 and a treatment site. As a non-limiting example, the treatment region(s) may include a plurality of electrodes 18 configured to deliver pulsed field electric energy to a tissue area in proximity to the electrodes 18. The medical device 12 may serve both as a treatment device and/or a mapping device. The medical device 12 may include an elongate body or shaft 22 passable through a patient's vasculature and/or proximate to a tissue region for diagnosis and/or treatment. For example, the medical device 12 may be a catheter that is deliverable to the tissue region via a sheath or intravascular introducer (not shown). The elongate body/shaft 22 may define a proximal portion 24, a distal portion 26, and a longitudinal axis 28, and may further include one or more lumens 27 disposed within the elongate body/shaft 22 thereby providing mechanical, electrical, and/or fluid communication between the elongate body proximal portion 24 and the elongate distal portion 26.

The medical device 12 may further include a handle 29 coupled to the elongate body proximal portion 24. The handle 29 may include circuitry for identification and/or use in controlling of the medical device 12 or another component of the system. Additionally, the handle 29 may also include connectors that are mateable to the energy supply 14 and/or the CEDS 16 to establish communication between the medical device 12 and the energy supply 14. The handle 29 may also include one or more actuation or control features that allow a user to control, deflect, steer, or otherwise manipulate a distal portion of the medical device 12 from the proximal portion of the medical device 12.

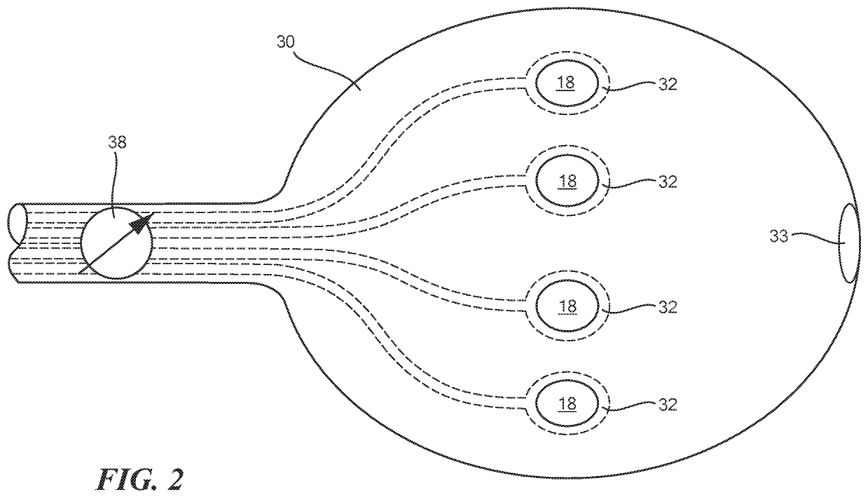

The medical device 12 may further include one or more expandable elements 30, coupled or affixed to, or otherwise disposed on the elongate body distal portion 26 for energetic, therapeutic, diagnostic and/or investigatory interaction between the medical device 12 and a treatment site or region. As a non-limiting example, expandable element 30 may include a balloon, such as the example as shown in FIGS. 1-2. In other examples, expandable element 30 may include other types of expandable elements including a basket structure, a combination of a basket structure and a balloon, or the balloon or combination of balloons that allow for the engagement, treatment, and/or diagnosis for varying anatomical tissue structures with different geometries and dimensions. The expandable treatment element 30 may include a basket structure with one or more deployable arms or splines that are movably coupled to the elongate body distal portion 26 and the one or more deployable arms or splines may include an electrically conductive surface to deliver and/or conduct electrical pulses to a designated treatment area. For example, each deployable arm or spline my include at least one electrode. The one or more deployable arms or splines may be movable from an expanded configuration to a contracted configuration and one or more deployable arms or splines may surround at least a portion of the circumference of the elongate body distal portion 26. In the expanded configuration, each spline and/or deployable arm may lie in a plane that is intersects the longitudinal axis of the elongate body/shaft 22 and in the retracted configuration the deployable arms or splines may be retracted within the elongate body/shaft 22 and/or in the retracted configuration the deployable arms or splines may be moved to create a loop in each deployable arm or spline and each deployable arm or spine is arranged as a set of overlapping or non-overlapping loops.

The medical device 12 may also include the plurality of electrodes 18 on the expandable element 30, for example, around or on an outer surface of the expandable element. The plurality of electrodes 18 may be any number and any size or shape. In one configuration, each of the plurality of electrodes 18 are coated with an anti-thrombogenic component to prevent blood clots form forming on the surface of the electrodes 18. In another configuration, a perimeter of each of the plurality of electrodes 18 has a higher thermal conductivity or lower electrical conductivity compared to a remainder of the each one of the plurality of electrodes to reduce edge effects and heating. Further examples of electrode 18 configurations may be found in U.S. Patent Publication Number 2019/0030328 the entirety of which is expressly incorporated by reference herein.

The electrodes 18 may be composed of any suitable electrically conductive material(s), such as metal or metal alloys. In a non-limiting example, the plurality of electrodes 18 may be deposited or printed onto an outer surface of the expandable element 30, or may be integrated with the material of the expandable element 30. Additionally, or alternatively, the plurality of electrodes 18 may be adhered to, mounted to, affixed to, or otherwise disposed on an inner surface of the expandable element 30A or on the outer surface of the expandable element 30B. In one embodiment, the medical device 12 may include a first expandable element 30A located within a second expandable element 30B (for example, as shown in FIG. 1). In this configuration, one or more electrodes 18 optionally may be located within an interstitial space 31 between the first 30A and second 30B expandable elements.

Referring now to FIGS. 1-3, the expandable element 30 may define or otherwise include a one or more irrigation channels 32 on the surface of or disposed within the balloon 30 and in communication with a fluid source 34. At the distal end of the expandable element 30 may be a distal end and the distal end may be a distal electrode 33. The one or more irrigation channels 32 may be closed or open channels that direct fluid or irrigant 36 from the fluid source 34 to preselected electrodes 18. For example, each electrode 18 in the plurality of electrodes may define an irrigation channel 32 around partially or the entirety of the perimeter of each electrode 18. Each channel 32 may be fluidly coupled to the controller 11 such that each channel 32 may be selectively activated to irrigate a desired electrode 18.

For example, as shown in FIG. 2, in a configuration in which there are four electrodes 18 about the expandable element 30, four irrigation channels 32 may be included. Each irrigation channel 32 may be independently fluidly coupled to the fluid source 34 for selective irrigation. Each irrigation channel 32 may be associated with at least one electrode 18. The flow of irrigant may be increased or decreased within each irrigation channel 32, and this increase or decrease of the flow or irrigant may be monitored by a flow meter 38 disposed within the medical device 12 and the flow meter could provide information about the flow rate of the irrigant. In one embodiment, the flow meter 38 may be disposed in the distal portion 26 of the shaft 22. Irrigant 36 may flow through each irrigation channel 32 to each electrode 18 at different rates as well as ad different times depending on the setting of the controller 11 which is configured to modulate flow of the irrigant 36. In this way, electrodes 18 may be selectively irrigated (e.g., irrigation controlled on an individual electrode basis), based on a variety of factors such as to achieve a desired flow rate, a particular timing, or other factors (e.g., electrode temperature).

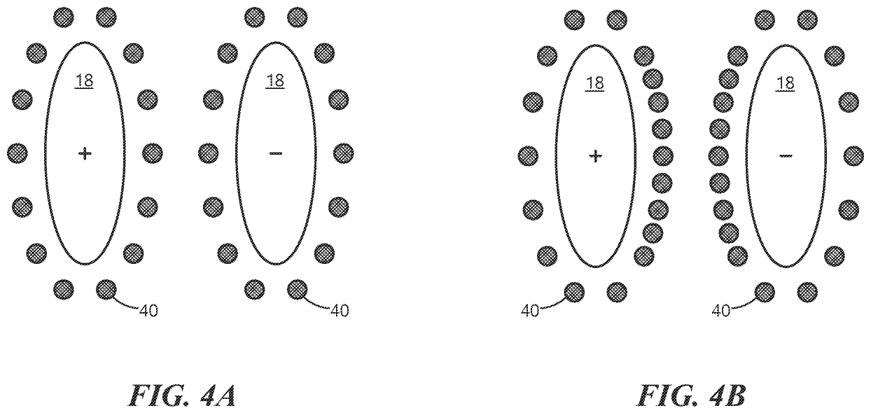

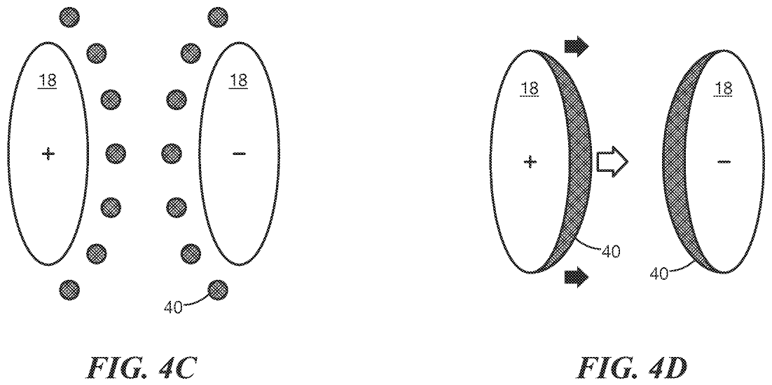

Additionally or alternatively, pressure sensing elements, thermal dilution monitoring with temperature probes, and/or flow transducers may be used in the medical device 12 and these components could be in communication with the irrigation control system 42 to provide information and feedback about the flow or irrigant 36. Integration of valves or passive flow correction mechanisms may be placed in or near the irrigation control system 42 to provide information about the flow of irrigant 36 within the medical device 12. The control of the flow of the irrigant 36 within the system 10 may be controlled in a variety of different ways. For example, each irrigation channel 32 may be controlled by a manifold that can breakout to individual valves or restrictors for each irrigation channel 32 and the irrigant 36 can come from one or more common sources. The flow meter 38 may continually or periodically monitor the flow in the one or more irrigation channels 32. The irrigant 36 comes from the irrigant 36 provided by the irrigation control system 42. The irrigant 36 may move through the irrigation channels 32 and directly interfaces with the electrodes 18 and tissue near the electrodes 18. The electrodes 18 may not be disposed within the expandable element 30, but may be disposed on the surface of the expandable element 30. Furthermore, any wiring associated with the electrodes 18 would be separate from the irrigation channels 32. As shown in FIGS. 4-7, the irrigation channels 32 may not be colinear and they may be in separate and distinct locations from one another on the expandable element 30. When there are multiple irrigation channels 32, certain sets of irrigation channels 32 may be linked together or broken out from fewer direct channels to the irrigation control system 42. For example, in one exemplary embodiment, there may be two irrigation channels 32 coming from the irrigation control system 42 and go through the one or more umbilicals 41. The flow of any fluid and/or irrigant 36 through these irrigation channels 32 may be monitored by the flow meter 38 before the irrigant 36 moves into the irrigation channels 32 which are associated with certain positive and negative electrodes 18. Each of these irrigation channels 32 are in direct communication with the one or more umbilicals 41.

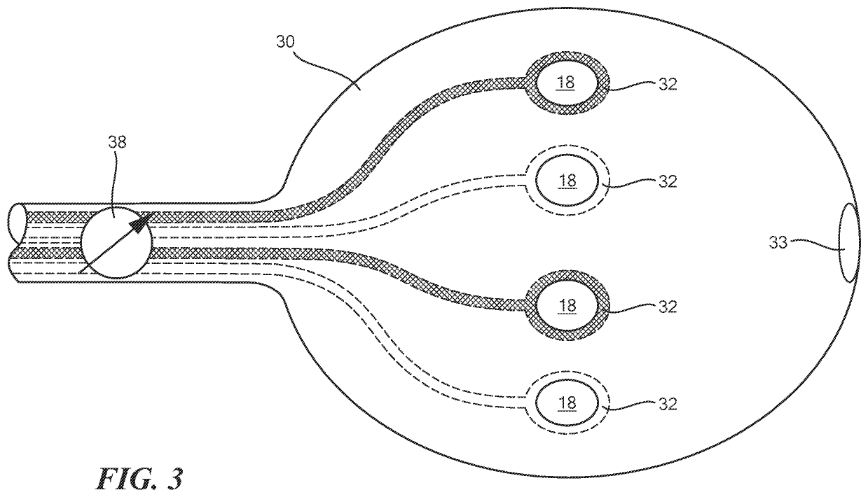

In another configuration, as shown in FIG. 3, the first and third electrodes 18 are irrigated, but the second and fourth electrodes are not. As shown in FIG. 3, each irrigation channel 32 surrounds the entirety of the perimeter of each electrode 18 in this configuration. The controller 11 may be used to select specific irrigation channels 32 to deliver fluid or irrigant 36 to. For example, the controller may close the second and fourth irrigation channels 32 to restrict the flow of fluid or irrigant 36 and open the first and the third irrigation channels 32 to allow fluid or irrigant 36 to flow into these particular irrigation channels 32. The flow meter 38 may be in communication with the controller 11 to increase or decrease the flow of the irrigant 36 to particular irrigation channels 32. In the configuration as shown in this embodiment, the first and third irrigation channels 32 may be opened by the flow meter 38 to allow irrigant 36 to readily flow into each of these irrigation channels 32. The flow meter 38 may also be in communication with the controller 11 and close the second and fourth irrigation channels 32 to restrict the flow of irrigant 36 to each of these irrigation channels 32. In one configuration, the irrigation channels 32 are defined on the outer surface of the inner balloon 30A, but may optionally be on the outer balloon 30B. Additionally or alternatively, the irrigation channels 32 as well as the electrodes 18 may be found in the interstitial space 31 between the inner balloon 30A and the outer balloon 30B. The controller 11 may be configured to modulate the flow of the irrigant 36 to the one or more irrigation channels 32 to irrigate select electrodes 18 based upon a variety of different parameters. For example, the controller 11 may be configured to modulate the flow of the irrigant 36 to the one or more irrigation channels 32 to select electrodes 18 based upon whether or not the select electrodes 18 are activated or deactivated. In one configuration, irrigant 36 may only flow to electrodes 18 that are activated during delivery of PEF energy to tissue. The remaining electrodes 18 that are not activated may not be irrigated.

Referring to FIG. 1, in one configuration, the handle 29 may have one or more umbilicals and each umbilical 41 on the handle 29 may fluidly couple the medical device 12 as well as with the fluid source 34. The umbilicals 41 may also be in communication with an irrigation control system 42. The irrigation control system 42 may include a variety of different components including integrated sensors such as a flow meter, temperature sensor, etc., which may modulate the flow of irrigant to the handle 29. The medical device 12 may modulate the flow of fluid to the handle 29 and then into the shaft 22 as well as additional modulation of the fluid flow with the flow meter 38 which may be disposed on the distal portion of the shaft 22. The irrigation control system 42 may be in communication with the flow meter 38 and use certain preset parameters from the flow meter 38 to control the flow of irrigant 36 within the system 10. Alternatively, the system may include separate flow meters for any irrigation channels 32 that the flow meter 38 is controlling. In one embodiment, the irrigation control system 42 could be set to provide a constant flow rate of irrigant 36 to one or more than one irrigant channel 32 to provide the desired flow level in each respective irrigation channel. Alternatively, the system 10 could maintain a constant and low flow rate of irrigant 36 to continuously keep each irrigation channel 32 open while the system 10 is in communication with the energy supply 14 and the processing circuitry 13. The system 10 may be configured to provide a higher flow rate of irrigant 36 when energy is being delivered to the electrodes 18 and the rages and configurations specific to the particular energy level being delivered to each electrode 18 as well as the type of energy being delivered. For example, the processing circuitry 13 may be set to recognize when energy delivery to the electrodes 18 is going to happen and when it is going to be completed. Accordingly, the flow of irrigant 36 to the irrigant channels 32 may be based upon the energy delivery cycle to the electrodes 18. The irrigation control system 42 may also be configured to be in communication with the energy supply 14 to know what level of energy is going to be delivered to the electrodes 18 and the irrigant 36 that is delivered to the particular irrigation channels may have a particular temperature, conductivity, or may be mixed from multiple fluid sources to help with the safety and efficacy of the delivery of energy to tissue.

In one configuration, the fluid source 34 is included with the controller 11 as part of a common controller. In other configurations the fluid source 34 is separate and distinct from the controller 11. The fluid or irrigant 36 within the fluid source 34 may be any kind of irrigant 36 and the irrigant 36 may be temperature controlled by the fluid source 34. The fluid source 34 may include a heating element or a cooling element as well as a temperature sensor to control the temperature of the irrigant 36 within the fluid source 34. The controller 11 may control the temperature setting within the fluid source 34 so that the irrigant 36 may be heated or cooled to a specific temperature within the fluid source 34. For example, the temperature of the irrigant 36 may be set by the controller 11 and once the irrigant 36 gets to a particular preset temperature the irrigant 36 may flow from the fluid source 34 at the preset temperature. The preset temperature may be a temperature that is less than an ambient temperature of blood to cool the tissue being treated and/or the particular electrode 18 being irrigated. Moreover, the irrigant 36 may be saline, composed of about half saline, may have a lower or higher conductivity than blood, may be visible under imaging such as fluoroscopy or MRI, may be heparinized to prevent coagulation on the electrodes 18, may include a least two different types of fluids, may have a net negative charge to reduce a risk of coagulation formation at the plurality of electrodes and/or may be configured to increase a vulnerability of the tissue to PEF energy. The irrigant 36 may be visible under imaging such as fluoroscopy or MRI and including a contrast media. The contrast media may be made from a liquid that temporarily changes the way imaging tools interact with the body but do not permanently discolor internal organs and do not produce radiation. The contrast media may make certain structures or tissue within the body appear different on the images than they would if no contrast media were administered and this may assist in the visibility of certain tissues, blood vessels or organs. The contrast media may include iodine-based and barium sulfate compounds, barium-sulfate, gadolinium, saline, and gas.

In some configurations, another parameter that the controller 11 may be configured to use to modulate the flow of irrigant 36 to the electrodes 18 is based on one or more PEF ablation parameters. The flow of irrigant 36 may be increased or decreased based upon certain preset PEF ablation parameters. This increase or decrease of flow may be further monitored by the flow meter 38 disposed within the medical device 12, depending on the desired lesion characteristic. For example, irrigant 36 composed of components that increase the vulnerability of tissue to PEF energy may be initiated by controller 11 and may be based on parameters derived from prior deliveries of PEF energy to the tissue which may include at least one from the group consisting of: temperature rise at the electrode, impedance change at or between electrodes, quantity of fluid delivered, pressure of the irrigation channel, measured flow rate, change in delivered current over the period of PEF delivery, and total energy expenditure of energy source for PEF energy delivery. Similarly, the controller 11 may be further configured to modulate an amount of fluid delivered to the one or more irrigation channels 32 based at least in part on preselected PEF ablation parameters which may include at least one from the group consisting of applied voltage, pulse width, cycle lengths, number of applied pulses per application, number of applications, and selection of PEF energy delivering elements.

Referring now to FIGS. 4A-4D are various configurations of electrodes 18 in relation to one or more fluid ports 40. Each fluid port 40 is in communication with the one or more irrigation channels 32 to irrigate at least a portion of the electrodes 18, which may have a positive polarity as indicated by a “+” or a negative polarity as indicated by a “−”. In the figures, the “+” and “−” polarities are not indicated to mean that the polarities are always permanent but rather to illustrate that bipolar energy is transferred between opposing polarities, that may be fixed or variable. The circular and oval electrodes 18 in the figures are showed to demonstrate various examples of how irrigation channels 32 may be placed around the edges of the electrodes 18 and how this may be influenced by opposing polarity elements. The electrodes 18 may also have different shapes and be placed in different location within the medical device 12.

For example, as shown in FIG. 4A, a plurality of ports 40 may be symmetric about each of a pair of electrodes 18 to evenly irrigate the same. Each electrode 18 from the pair of electrodes may have an opposite polarity and each electrode 18 may be symmetrically surrounded by the plurality of ports 40. As shown in FIG. 4A, the positively charged electrode 18 has fourteen separate ports 40 and the negatively charged electrode 40 has fourteen separate ports 40. The ports 40 may each be in communication with an irrigation channel 32. Each port 40 may be in communication with a different irrigation channel 32 or in an alternative embodiment more than one port 40 may be in communication with an irrigation channel 32. The symmetric ports 40 around each electrode 18 may allow for the even distribution of the irrigant 36 around each electrode 18.

As shown in FIG. 4B, the plurality of ports 40 may also asymmetrically surround each electrode 18 and the ports 40 may be concentrated on the side of each electrode 18 facing the other electrode 18 to allow increased irrigation in the portions where there are more ports 40. As shown in FIG. 4B, there may be eighteen ports 40 surrounding each electrode 18 and half of the ports 40 may be on one side of each electrode 18. The number of ports 40 used may depend upon the dimensions of the electrode 18 being surrounded by the ports 40. The highest concentration of the ports 40 may be found at the points where the distance between the opposing polarities of each of the electrodes 18 is shortest. These are the likely location of increased current and consequently heating, where the increased number of ports and consequently flow can be most effective. Accordingly, a higher concentration of ports 40 may occur as proportionally a greater amount of irrigant 36 may be needed in these areas to sufficiently cool the electrodes 18.

As shown in FIG. 4C the ports 40 may be positioned on a single side of each electrode 18. In this embodiment, one side of the electrode 18 may have seven ports 40. These ports may be found on the side of the electrode 18 where the distance between the opposing polarities of each of the electrodes 18 is the shortest and ports are absent from the portions of the electrodes which are not in sufficient proximity to an opposing polarity. This may allow for the cooling of tissue or the area surrounding each electrode 18 where the most heating may occur when the electrode 18 is delivering energy to tissue or an area of the body while limiting the volume required for irrigation by not using one or more irrigant ports where they are of less utility.

In FIG. 4D, a single port 40 may be disposed on an entire side of each electrode 18. The port 40 may have a uniform thickness or a variable thickness. As shown in FIG. 4D, the port 40 may be in contact with or have a portion of its perimeter defined by the electrode 18. The side of the electrode 18 where the distance between the opposing polarities is each of the electrodes 18 is the shortest. Having one larger port 40 on one side of each electrode 18 may significantly cool the area surrounding the port 40 to avoid overheating damage to tissue when energy is being delivered to the electrode 18 as well as after energy has been delivered to the electrode 18. This configuration may allow irrigant 36 to be delivered directly to each electrode 18 including the edge of the electrode 18 where the port 40 is disposed. The at least one port 40 may only be on one side of each electrode 18 or the at least one port 40 may be found surrounding the entirely of the perimeter of the electrode 18.

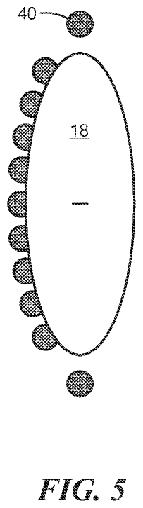

Referring now to FIG. 5, the plurality of ports 40 may be on a single side of the electrode 18 and there may be a port on the distal portion and the proximal portion of the electrode 18 as well. This configuration of the ports 40 may at least partially cover a portion of the electrode 18 such that irrigant is delivered directly to the edge of the electrode 18. Having the ports 40 that are at least partially covering a portion of the electrode 18 can allow irrigant 36 to be delivered to the tissue that is in close proximity to the electrode 18. This can allow for the delivery of irrigant 36 to the tissue that may experience the most heating when energy is delivered to the electrode 18.

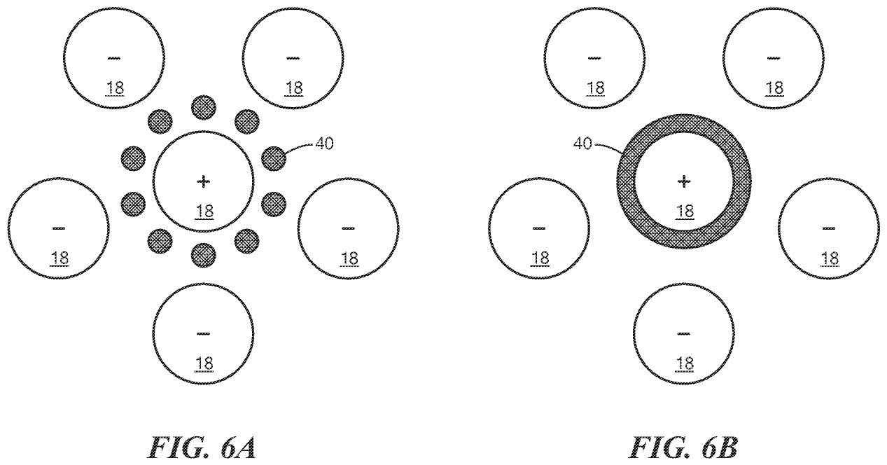

As shown in FIGS. 6A and 6B, the ports 40, which may be a plurality of ports 40 or a single port 40 may circumscribe the electrode 18. The ports 40 may be disposed near and/or around the electrode 18 with the positive polarity as this electrode 18 may have a greater intensity in the electrical field due to the negative polarity of the surrounding electrodes 18. As shown in FIG. 6A there are a plurality of electrodes 18 that are in close proximity to one another. In this exemplary configuration, there is one electrode 18 that has a positive polarity and the electrodes 18 surrounding the one electrode 18 all have a negative polarity. The electrode 18 with the positive polarity may be surrounded by a plurality of ports 40 that circumscribe the electrode 18. In this configuration, there are ten ports 40 that surround the electrode 18. The ports 40 may symmetrically surround the electrode 18 or the ports 40 may be configured to asymmetrically surround the electrode 18. This configuration may allow irrigant 36 to be delivered to each of the ports 40 simultaneously or irrigant may be delivered to certain select ports 40 at certain times. The delivery of irrigant 36 to the ports 40 may be based upon certain preset parameters within the controller 11. In FIG. 6B, the electrode 18 with the positive polarity has a port 40 which surrounds the entirety of the electrode 18. The port 40 which circumscribes the entirety of the electrode 18 may allow for the even distribution of irrigant 36 around the electrode 18, for example, when energy is being delivered to the electrode 18 or after the delivery of energy. This can prevent damage to tissue that is in proximity with the electrode 18 with the positive polarity. Placement of negative polarity electrodes 18 in FIGS. 6A and 6B are to illustrate the role of proximity to opposing polarities for the positive electrode 18 but may themselves be similarly augmented with irrigation ports 40.

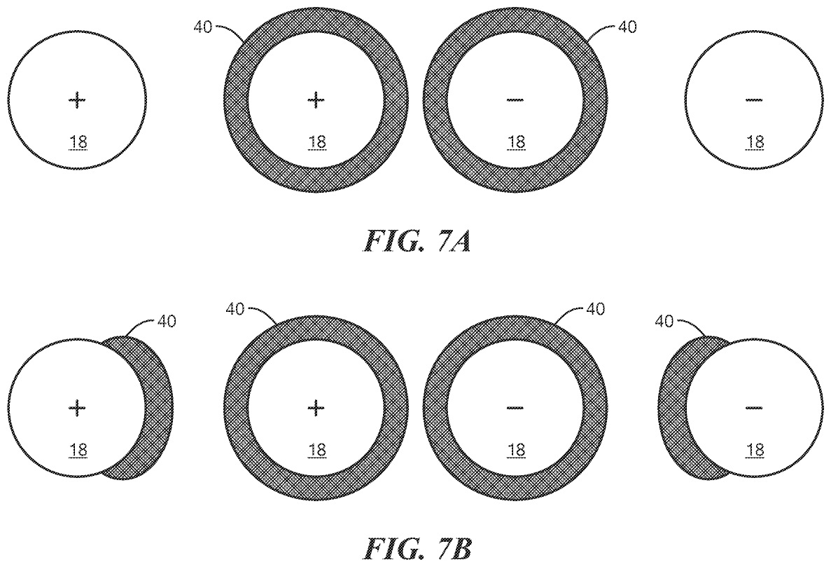

As shown in FIGS. 7A and 7B, the electrodes 18 with opposite polarities may include a greater number of ports 40 than those electrodes with the same polarity. In FIG. 7A, there are two electrodes 18 that have opposite polarities which are in close proximity to one another. These two electrodes 18 with the opposite polarities may have a port 40 which entirely surrounds each of these electrodes 18. This electrode 18 may have a greater intensity in the electrical field due to the negative polarity and the positive polarity of the surrounding electrodes 18. Having the ports 40 which fully surround these electrodes 18 may help deliver the irrigant 36 to the area surrounding these electrodes 18. Additionally, as shown in FIG. 7A, there is an electrode 18 with positive polarity near the electrode 18 with the positive polarity that is surrounded by the port 40 but this electrode 18 does not have any ports 40 surrounding it. This may occur when there are electrodes 18 with the same polarity that are near one another. Also, there is an electrode with the negative polarity which is surrounded by the port 40 and the other electrode 18 which also has the negative polarity that is in close proximity does not have any ports 40. These two electrodes have the same polarity and are in close proximity to one another and therefore may not require irrigant 36 to be delivered to both electrodes 18.

Now referring to FIG. 7B, there are four electrodes 18 shown with two having the positive polarity and two having the negative polarity. In this configuration, the electrodes 18 which have the positive polarity and the negative polarity and are located next to one another have the ports 40 which surround the entirety of each electrode 18. Additionally, there is the electrode 18 with the positive polarity located next to the other electrode 18 with the positive polarity. This second electrode 18 with the positive polarity may have the port 40 which at least partially surrounding the electrode 18 substantially targeting the portion of the electrode in the direction of opposing polarity elements where electrical current and heat may be increased. This configuration can provide irrigant 36 to the tissue that is near and surrounding both of the electrodes 18 with the positive polarity. There may also be the electrode 18 with the negative polarity located next to the other electrode 18 with the negative polarity. This second electrode 18 with the negative polarity may have the port 40 which at least partially surrounding the electrode 18. This configuration can provide irrigant 36 to the tissue that is near and surrounding both of the electrodes 18 with the negative polarity. This configuration can provide irrigant 36 to the tissue that is near and surrounding both of the electrodes 18 with the negative polarity. This additional irrigation of elements located further from opposing polarity electrodes may for example be advantageous if a relatively larger energy was delivered as compared with what may be mitigated using the configuration of FIG. 7A. The higher the energy delivery is to the electrodes 18 could cause an increase in the heating and edge effects in and around the electrode 18. It is helpful to mitigate the heating and edge effects such that the ports 40 may be used to help with cooling and allow for the delivery of more energy to the electrodes 18 so that any treatment may be more effective and efficient. The temperature of each electrode 18 may also be controlled using thermocouples (not shown) to modulate the flow of irrigant 36 into each irrigation channel 32.

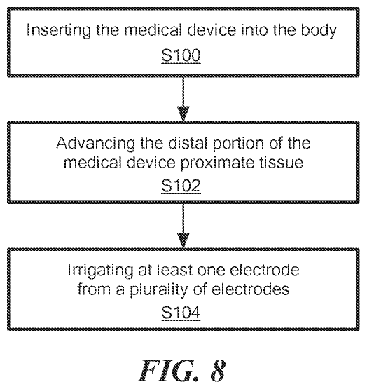

Now referring to FIG. 8, energy may be delivered to tissue using the medical device 12. It will be understood that various types of energy may be one or more energy modalities delivered to the medical device 12. Energy, including pulsed electric field (“PEF”) energy or radiofrequency (RF) energy may be delivered to tissue within the body using the medical device 12. The medical device 12 may be inserted into the body of the patient. S100. For example, the medical device 12 may be inserted into the tissue. A distal portion 44 of the medical device 12 may be advanced proximate certain tissue within or outside of the body. S102. The distal portion 44 of the medical device 12 may include various components including the expandable element or balloon 30, the plurality of electrodes 18, and the plurality of irrigation channels 32. The medical device 12 may include an expandable element or balloon 30 as part of the distal portion 44 of the medical device 12. The expandable element or balloon 30 may be on the distal portion 44 of the medical device 12 and the plurality of electrodes 18 may be disposed on the outer surface of the expandable element or balloon 30. Alternatively, the medical device may include at least two expandable elements 30 and with an outer expandable element and an inner expandable element 30. The plurality of electrodes 18 may be disposed in the interstitial space 31 which is between the at least two expandable elements 30. The plurality of electrodes 18 may be configured to deliver one or more than one energy modality which include the delivery of PEF energy. The plurality of irrigation channels may be disposed around at least the perimeter of each electrode 18 from the plurality of electrodes 18. At least one electrode 18 from the plurality of electrodes 18 may be selectively irrigated with the irrigant 36. All of the electrodes 18, one of the electrodes 18, or a select number of electrodes 18 may be irrigated with the irrigant 36 through the irrigation channels 32.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A medical device configured to deliver energy to tissue, comprising:
an elongated shaft having a proximal portion and a distal portion;
an expandable element coupled to the distal portion of the elongated shaft, the expandable element having an outer surface and an inner surface opposite the outer surface;
a plurality of electrodes disposed on the outer surface of the expandable element and configured to apply energy to tissue; and
the expandable element comprising one or more irrigation channels proximate to or on the plurality of electrodes, the one or more irrigation channels being configured to irrigate at least one of the plurality of electrodes.

2. The device of claim 1, wherein the one or more channels are disposed around a perimeter of each electrode from the plurality of electrodes.

3. The device of claim 1, wherein an irrigant is disposed within the one or more channels and the irrigant only flows toward the electrodes which are energized during the delivery of energy to tissue.

4. The device of claim 3, wherein the irrigant is cooler than an ambient temperature of blood.

5. The device of claim 3 wherein the irrigant has a lower conductivity than blood.

6. The device of claim 3 wherein the irrigant has a higher conductivity than blood.

7. The device of claim 1, wherein a perimeter of each of the plurality of electrodes has a higher thermal conductivity compared to a remainder of the each one of the plurality of electrodes to reduce edge effects and heating.

8. The device of claim 1, wherein a perimeter of each of the plurality of electrodes has a lower electrical conductivity compared to a remainder of the each one of the plurality of electrodes to reduce edge effects and heating.

9. The device of claim 3, wherein the irrigant is a contrast media that is visible using medical imaging under ultrasound or fluoroscopy to confirm irrigation.

10. The device of claim 1, wherein the one or more channels are configured to selectively irrigate at least one of the plurality of electrodes based upon a desired flow rate, a particular timing, or an electrode temperature.

11. A medical system configured to deliver pulsed electric field (PEF) energy to tissue, comprising:
a medical device, including:
an elongated shaft having a proximal portion and a distal portion;
a balloon with an outer surface and an inner surface opposite the outer surface, the balloon being coupled to the distal portion of the elongated shaft;
a plurality of electrodes disposed on the outer surface of the balloon and configured to apply PEF energy to the tissue, each electrode having a perimeter; and
the balloon including one or more irrigation channels around the perimeter of each of the plurality of electrodes, the one or more irrigation channels being configured to selectively irrigate the plurality of electrodes; and
a fluid source in communication with the one or more irrigation channels a controller in communication with the fluid source and the medical device, the controller being configured to deliver PEF energy to the plurality of electrodes and to modulate a delivery of a fluid from the fluid source to the one or more irrigation channels based upon at least one preset parameters derived from prior deliveries of PEF energy to the tissue.

12. The system of claim 11, wherein the at least one preset parameters are derived from prior deliveries of PEF energy to the tissue include at least one from the group consisting of: temperature rise, impedance change, quantity of fluid delivered, pressure of the irrigation channel, measured flow rate, change in delivered current over a period of PEF delivery, and total energy expenditure of energy source for PEF energy delivery.

13. The system of claim 11, wherein the controller is further configured to modulate an amount of fluid delivered to the one or more irrigation channels based at least in part on preselected PEF ablation parameters.

14. The system of claim 13, wherein the preselected PEF parameters include at least one from the group consisting of applied voltage, pulse width, cycle lengths, number of applied pulses per application, number of applications, and selection of which ones of the plurality of electrodes are engaged in PEF delivery.

15. The system of claim 11, wherein the controller is further configured to modify a temperature of the fluid in the fluid source.

16. The system of claim 11, wherein the fluid source includes at least two type of fluids.

17. The system of claim 11, wherein the fluid in the fluid source has a net negative charge.

18. The system of claim 11, wherein the plurality of electrodes includes an anti-thrombogenic coating.

19. The system of claim 11, wherein the fluid in the fluid source is configured to increase a vulnerability of the tissue to PEF energy.

20. A method of delivering pulsed electric field (PEF) energy to tissue, comprising:
advancing a distal portion of a medical device proximate the tissue, the medical device including:
a balloon at the distal portion;
a plurality of electrodes disposed on an outer surface of the balloon and configured to deliver PEF energy;
a plurality of irrigation channels disposed around a perimeter of each of the plurality of electrodes; and
selectively irrigating at least one of the plurality of electrodes.
